(19) United States
(12) Patent Application Publication
(10) Pub. No.: US 2009/0092696 A1
(43) Pub. Date: Apr. 9, 2009

(54) Herbal combination omega oil aphrodisiac

(76) Inventors: Douglas Burke, Newport Beach, CA (US); Laila S. Nabulsi, Costa Mesa, CA (US)

Correspondence Address: Douglas Burke, 2507 Port Whitby, Newport Beach, CA, 92660, US

(21) Appl. No.: 11/973,498

(22) Filed: Oct. 9, 2007

(51) Int. Cl. A61K 36/00; A61K 36/296; A61P 15/00

(52) U.S. Cl. 424/773; 424/725; 424/777

(57) ABSTRACT

A method of enhancing the libido in an adult human comprising the administering of a composition, comprising an effective amount of an herbal aphrodisiac selected from the group consisting of those herbs, which are purported to enhance the libido in humans, and an effective amount of omega oils.

### BACKGROUND OF THE INVENTION

Lepidium meyenii or maca is an herbaceous, biennial plant or annual plant (some sources say a perennial plant) native to the high Andes of Bolivia and Peru. It is grown for its fleshy hypocotyl (actually a fused hypocotyl and taproot), which is used as a root vegetable and a medicinal herb. Its Spanish and Quechua names include maca-maca, maino, ayak chichira, and ayak willku. Maca is cultivated in the Andean region, especially Peru, where it is a food staple. As reported in Dini et al., (Dini A., Miguliuolo G, Rastrelli L, et al. Chemical composition of Lepidium meyenii, Food Chemistry 1994; 49:347-9) and Walker (Walker M, Effect of Peruvian Maca on hormonal functions. Townsend Letter for Physicians 1998; November: 18-21), the tuber is also used in folk medical traditions as a mood enhancer, as an antidepressant, and to promote wound healing. Dini et al. also reports that the chemical composition of Maca includes a balanced protein compared to carrot and potato protein.

Maca also contains a variety of trace minerals, including iron, copper and iodine as well as saponins and alkaloids.

A synopsis of a book by Gloria Chacon de Popivici, “La importanica de Lepidium peruvianum Chacon (Maca) en la Alimentacion y Salud del ser Humano y Animal 2,000 Ados Antes y Despues de Cristo y en el Siglo XXI”, describes an experiment in which rats given Maca powdered root had higher sperm production and motility rates than in control groups. The author is quoted as saying the alkaloids of Maca affect the pituitary and hypothalamus, and it acts on adrenals, pancreas and thyroid. Along these lines, anecdotal information is available suggesting that Maca alleviates perimenopausal and post menopausal symptoms, male impotence erectile dysfunction, and female and male sterility, healing bone fractures, osteoporosis, premature aging, and chronic fatigue.

Several books and reports describe Andean roots and tubers as crops and sources of food and beverage. See Soensen et al., (Soensen M, Gruneberg W J, Orting B et al. Andean Roots and Tubers: ahipa, arracacha, maca and yacon. Centro Internaciojoal de la Papa, Lima Peru, 1997—Abstract); Condesan, (Pocket guide to nine exotic Andean roots and tubers. Lima, Peru. 1997. Abstract); Comas et al, (Comas M, Miquel X, de la Torre M C, Arias G. Estudio bromatoglogico de la maca o paca (Lepdium meyenii). Alimentaria 1997, no. 286:85-90. Abstract); Toledo et al., (Toledo J, Dehal P, Jarrin F et al. Genetic variability of Lepidium meyenii and other Andean Lepidium species assessed by molecular markers. An. Botany 1998; 82:523-30. Abstract). However, based on the information in the Abstracts, none of these references discuss the response of male hormone levels to ingestion of Maca.

In addition to sugars and proteins, Maca contains uridine, malic acid and its benzoyl derivative, and the glucosinolates, glucotropaeolin and m-methoxyglucotropaeolin. The methanol extract of Maca tuber also contained (1R,3S)-1-methyltetrahydro—carboline-3-carboxylic acid, a molecule which is reported to exert many activities on the central nervous system (Piacente, Sonia; Carbone, V., Plaza, A., Zampelli, A. & Pizza, C. (2002). “Investigation of the Tuber Constituents of Maca (Lepidium meyenii Walp.)”. Journal of Agricultural and Food Chemistry 50 (20): 5621-5625. PMID 12236688). The nutritional value of dried Maca root is high, similar to cereal grains such as rice and wheat. It contains 60% carbohydrates, 10% protein, 8.5% dietary fiber, and 2.2% fats. Maca is rich in essential minerals, especially selenium, calcium, magnesium, and iron, and fatty acids including linolenic acid, palmitic acid, and oleic acids, and 19 amino acids, as well as polysaccharides (Muhammad, I; Zhao J., Dunbar D. C. & Khan I. A. (2002). “Constituents of Lepidium meyenii ‘maca’”. Phytochemistry 59 (1): 105-110. PMID 11754952). Maca's reported beneficial effects for sexual function could be due to its high concentration of proteins and vital nutrients (Chacón de Popovici, G. (1997). La importancia de Lepidium peruvianum (“Maca”) en la alimentacion y salud del ser humano y animal 2,000 anos antes y desputes del Cristo y en el siglo XXI. Lima: Servicios Gráficos “ROMERO”), though Maca contains a chemical called p-methoxybenzyl isothiocyanate, which reputedly has aphrodisiac properties.

Yohimbine, also known under the antiquated names quebrachin, aphrodin, corynine, yohimvetol and hydroergotocin, is the principal alkaloid of the bark of the West-African evergreen Pausinystalia yohimbe Pierre (formerly Corynanthe yohimbe) family Rubiaceae (Madder family). There are 31 other yohimbane alkaloids found in Yohimbine. In Africa, yohimbine has traditionally been used as an aphrodisiac.

Yohimbine is a selective, competitive, alpha2-adrenergic receptor antagonist that is sometimes used as an alternative treatment for erectile dysfunction (National Institutes of Health). The alpha2 receptor is responsible for sensing adrenaline and noradrenaline and telling the body to decrease its production as part of a negative feedback loop. Yohimbine supposedly acts as antagonist, or a blocker, by binding to alpha2 receptors, but not activating them. This in turn increases adrenal gland production of adrenaline and noradrenaline. Yohimbine also antagonizes several serotonin receptor subtypes: 1A (inhibitory, behavioral control), 1B (inhibitory, vasoconstriction), 1D (inhibitory, vasoconstriction), and 2B (smooth muscle contraction). Since yohimbine is an antagonist, it will decrease the effects of these receptors, thus causing excitation, vasodilation, and smooth muscle relaxation. Yohimbine is also said to increase dopamine and have some actions as an MAOI, although these mechanisms are unknown.

Higher doses of oral yohimbine may create numerous side effects such as rapid heart rate, high blood pressure, and overstimulation. Yohimbine might produce anxiety, and is thought to cause insomnia and sleeplessness in some users.

Some internet shops sell expensive formulations of yohimbine for transdermal delivery to effect a local reduction of adipose tissue, although there is no evidence that it is effective. Demand for products of this kind is frequently found in the bodybuilding community.

Yohimbine chloride, a standardized form of yohimbine, is a prescription medicine that has been used to treat erectile dysfunction (National Center for Complimentary and Alternative Medicine). Controlled studies suggest that it is not always an effective treatment for impotence, and evidence of increased sex drive (libido) is anecdotal only (http://pharmrev.aspetjournals.org/cgi/content/ful/53/3/417#SEC4_{—}4_{—}6_{—}2). It has significant side effects such as anxiety reactions. According to the Mayo Clinic, yohimbine can be dangerous if used in excessive amounts.

Serenoa repens, or saw palmetto, is the sole species currently classified in the genus Serenoa. It has been known by a number of synonyms, including Sabal serrulatum, under which name it still often appears in alternative medicine. It is a small palm, normally reaching a height of around 2-4 m. Its trunk is sprawling, and it grows in clumps or dense thickets in sandy coastal lands or as undergrowth in pine woods or hardwood hammocks. Erect stems or trunks are rarely produced, but are found in some populations. It is endemic to the southeastern United States, most commonly along the Atlantic and Gulf coastal plains, but also as far inland as southern Arkansas. It is extremely slow growing, and long lived, with some plants, especially in Florida, being over 700 years old.

Saw palmetto is a fan palm (Arecaceae tribe Corypheae), with the leaves having a bare petiole terminating in a rounded fan of about 20 leaflets. The petiole is armed with fine, sharp teeth or spines that give the species its common name. The leaves are light green inland, and silvery-white in coastal regions. The leaves are 1-2 m in length, the leaflets 50-100 cm long. They are similar to the leaves of the palmettos of genus Sabal. The flowers are yellowish-white, about 5 mm across, produced in dense compound panicles up to 60 cm long. The fruit is a large reddish-black drupe and is an important food source for wildlife. The plant is used as a food plant by the larvae of some Lepidoptera species including Batrachedra decoctor (which feeds exclusively on the plant).

The genus name honors American botanist Sereno Watson.

The fruits of the Saw Palmetto are highly enriched with fatty acids and phytosterols, and extracts of the fruits have been the subject of intensive research for the treatment of urinary tract infections.

Saw palmetto extract is an extract of the fruit of Serenoa repens. It is rich in fatty acids and phytosterols, and has been shown to be effective in the management of benign prostatic hyperplasia (Wilt T J et al (1998). “Saw palmetto extracts for treatment of benign prostatic hyperplasia: a systematic review”. JAMA 280: 1604-1609).

Native Americans used the fruit for food, but also in the treatment of a variety of urinary and reproductive system problems. The European colonists learned of the use of Saw Palmetto. It was used as a crude extract for at least 200 years for various conditions including asthenia (weakness), recovery from major illness, and urogenital problems. For instance, the Eclectic physician H. W. Felter wrote of it, “Saw palmetto is a nerve sedative, expectorant, and a nutritive tonic, acting kindly upon the digestive tract. Its most direct action appears to be upon the reproductive organs when undergoing waste of tissue.” (Felter's Complete Text: http://www.henriettesherbal.com/eclectic/felter/serenoa.html).

The Eclectics knew Saw Palmetto as more than a prostate herb. King's American Dispensatory, in 1898 claims:
It is also an expectorant, and controls irritation of mucous tissues. It has proved useful in irritative cough, chronic bronchial coughs, whooping-cough, laryngitis, acute and chronic, acute catarrh, asthma, tubercular laryngitis, and in the cough of phthisis pulmonalis. Upon the digestive organs it acts kindly, improving the appetite, digestion, and assimilation. However, its most pronounced effects appear to be those exerted upon the urino-genital tracts of both male and female, and upon all the organs concerned in reproduction. It is said to enlarge wasted organs, as the breasts, ovaries, and testicles, while the paradoxical claim is also made that it reduces hypertrophy of the prostate. Possibly this may be explained by claiming that it tends toward the production of a normal condition, reducing parts when unhealthily enlarged, and increasing them when atrophied (King's American Dispensatory 1898: http://www.henriettesherbal.com/eclectic/kings/serenoa.html).

In modern times, much research has been done on extract made from the fruits, which are highly enriched with fatty acids and phytosterols. This research has been the subject of a thorough meta-analysis published in the Journal of the American Medical Association and has been shown to be effective for the treatment of men with symptomatic benign prostatic hyperplasia (enlargement of the prostate) compared to placebo and the two major categories of drugs used for men with this condition (Wilt T J et al (1998). “Saw palmetto extracts for treatment of benign prostatic hyperplasia: a systematic review”. JAMA 280: 1604-1609). There are also small, positive clinical trials published on the use of Saw Palmetto extracts topically and internally for male-pattern baldness. In 2005, a long-term, placebo-controlled trial showed that a combination of Saw Palmetto fruit and nettle root extracts were effective in treating urinary tract symptoms in older men (Lopatkin N et al (2005). “Long-term efficacy and safety of a combination of sabal and urtica extract for lower urinary tract symptoms—a placebo-controlled, double-blind, multicenter trial”. World Journal of Urology 23 (2): 139-146). However, in February 2006, a large, blinded placebo-controlled study published in the New England Journal of Medicine showed no reduction of symptoms from enlarged prostate by taking Saw Palmetto, as compared to placebo (Bent S et al (2006). “Saw Palmetto for Benign Prostatic Hyperplasia”. NEJM 354: 557-56). Designers of the latest study questioned whether the differently flavored placebos in previous studies were adequately blinded. Critics of the latest study questioned whether a sufficient dosage of active ingredients was given (Allison Aubrey. Morning Edition: Study Casts Doubt on Saw Palmetto as Prostate Remedy [Audio recording]. National Public Radio). An earlier single case study on saw palmetto concluded that searching for information on a herbal medicine using MEDLINE alone was insufficient, and expanded their search to “alternative” databases, including AGRICOLA, EMBASE, IBIS, and Cochrane, plus a manual search of unindexed herbal journals (McPartland J M, Pruitt P L. (2000). “Benign prostatic hyperplasia treated with saw palmetto: a literature search and an experimental case study”. JAOA 100 (2): 89-96).

Other research has shown that the herb works by multiple mechanisms, including inhibiting 5alpha-reductase, interfering with dihydrotestosterone binding to the androgen receptor, by relaxing smooth muscle tissue similarly to alpha antagonist drugs, and possibly by acting as a phytoestrogen (Di Silverio F et. al (1998). “Effects of long-term treatment with Serenoa repens (Permixon) on the concentrations and regional distribution of androgens and epidermal growth factor in benign prostatic hyperplasia”. Prostate 37 (2): 77-83), (Plosker G L, Brogden R N (1996). “Serenoa repens (Permixon). A review of its pharmacology and therapeutic efficacy in benign prostatic hyperplasia”. Drugs Aging 9 (5): 379-95).

Because the fruit is the part used, and a prolific quantity is produced by an adult Saw Palmetto plant, this herbal medicine is considered ecologically sustainable.

Though in vitro studies suggest Saw Palmetto has properties that might make it useful against prostate cancer cells or to reduce prostatitis, clinical trials are lacking.

David Winston RH (AHG) describes a variety of conditions where Saw Palmetto extract is useful for men and women, using both research and ethnobotanical uses (David Winston (1999). Saw Palmetto for Men & Women: Herbal Healing for the Prostate, Urinary Tract, Immune System and More (Medicinal Herb Guide). Storey Publishing, LLC. ISBN 978-158017206).

Though men taking Saw Palmetto may develop mild nausea, reduced libido, or erectile dysfunction, the rate of such problems is clinically and statistically far less common than in men taking drugs to treat BPH symptoms, based on the JAMA meta-analysis cited above. There are no known drug interactions. It should generally be avoided in pregnancy and lactation and in small children due to lack of experience and knowledge in these populations and because of the purely theoretical risk of hormonal interference.

While Saw Palmetto is generally considered safe, one of its primary active ingredients, beta-sitosterol, is chemically similar to cholesterol. High levels of sitosterol concentrations in blood have been correlated with increased severity of heart disease in men who have previously suffered from heart attacks (Assmann G, et. al (2006). “Plasma sitosterol elevations are associated with an increased incidence of coronary events in men: results of a nested case-control analysis of the Prospective Cardiovascular Munster (PROCAM) study”. Nutr Metab Cardiovasc Dis 16 (1): 13-21).

For some people, an increase in libido has been reported through usage. As with other nutrients and herbs, various people will have different responses based on their chemical and biological make up.

Since Saw Palmetto has been known to have side effects, men taking it may develop mild nausea, reduced libido, or erectile dysfunction, it would be unobvious to combine it in a formula to enhance libido in men. This is a critical fact from the prior, which builds a case for one of the unobvious inventive steps of the present invention.

The side effect of causing flaccidity in the penis is expected since Saw Palmetto enhances urinary flow. Thus any substance that aided in urinary flow would not be put in an aphrodisiac mixture for men, since it would promote erectile dysfunction as opposed to enhancing the libido.

Omega-3 fatty acids are a family of polyunsaturated fatty acids, which have in common a carbon-carbon double bond in the ω-3 position.

Important nutritional essential omega-3 fatty acids are: α-linolenic acid (ALA), eicosapentaenoic acid (EPA), and docosahexaenoic acid (DHA). For a more complete list see List of omega-3 fatty acids. The human body cannot synthesize omega-3 fatty acids de novo, but it can form 20- and 22-carbon unsaturated omega-3 fatty acids from the eighteen-carbon omega-3 fatty acid, α-linolenic acid. These conversions occur competitively with omega-6 fatty acids, which are essential closely related chemical analogues that are derived from linoleic acid. Both the omega-3 α-linolenic acid and omega-6 linoleic acid are essential nutrients, which must be obtained from food. Synthesis of the longer omega-3 fatty acids from linolenic acid within the body is competitively slowed by the omega-6 analogues. Thus accumulation of long-chain omega-3 fatty acids in tissues is more effective when they are obtained directly from food or when competing amounts of omega-6 analogs do not greatly exceed the amounts of omega-3.

Omega-6 fatty acids are fatty acids where the term “omega-6” signifies that the first double bond in the carbon backbone of the fatty acid, occurs in the omega minus 6 position; that is, the sixth carbon from the end of the fatty acid.

The biological effects of the ω-6 fatty acids are largely mediated by their interactions with the ω-3 fatty acids.

Linoleic acid (18:2), the shortest chain omega-6 fatty acid is an essential fatty acid. Arachidonic acid (20:4) is a physiologically significant n-6 fatty acid and is the precursor for prostaglandins and other physiologically active molecules.

Some medical research has suggested that excessive levels of omega-6 acids, relative to omega-3 fatty acids, may increase the probability of a number of diseases and depression. Modern Western diets typically have ratios of omega-6 to omega-3 in excess of 10 to 1, some as high as 30 to 1. The optimal ratio is thought to be 4 to 1 or lower (http://www.csuchico.edu/agr/grsfdbef/health-benefits/ben-o3-o6.html).

Omega-9 fatty acids are a class of unsaturated fatty acids which have a C═C double bond in the ω-9 position. Some ω-9's are common components of animal fat and vegetable oil.

Two commercially important co-9 fatty acids are:
Oleic acid (18:1 ω-9), which is a main component of olive oil and other monounsaturated fats.
Erucic acid (22:1 ω-9), which is found in rapeseed, wallflower seed, and mustard seed. Rapeseed with high erucic acid content is grown for commercial use in paintings and coatings as a drying oil.

Unlike ω-3 and ω-6 fatty acids, ω-9 fatty acids are not classed as essential fatty acids (EFA). This is both because they can be created by the human body from unsaturated fat and are therefore not essential in the diet, and because the lack of an ω-6 double bond keeps them from participating in the reactions that form the eicosanoids.

Under severe conditions of EFA deprivation, mammals will elongate and desaturate oleic acid to make mead acid, (20:3 ω-9). (Lipomics) This also occurs to a lesser extent in vegetarians and semi-vegetarians. (Phinney, 1990)

The fatty acids are not known to have any aphrodisiac effects on humans.

Hemp oil is from the seed of the hemp plant that contains between 25-35% oil by weight, which is high in essential fatty acids. Cold-pressed, unrefined hemp oil is light green, with a nutty, grassy flavor.

Refined hemp oil is clear with little flavor. It is widely used in body care products, lubricants, paints and industrial uses. Antimicrobial properties make it a useful ingredient for soaps, shampoos and detergents. The oil is of high nutritional value because its 3:1 ratio of omega-6 to omega-3 essential fatty acids matches the balance required by the human body (InnVista, November 2005). It has also received attention in recent years as a possible source of bio-diesel (Agua Das (Nov. 16, 1997). Hemp Oil Fuels & How to Make Them. HempFarm.com). There are a number of organizations that promote the production and use of hemp oil (http://www.hempworld.com/pstindex0.htm).

Hemp oil is deliberately manufactured to contain no significant amounts of THC and is therefore, not a psychoactive drug.

Hemp oil is not known to have any aphrodisiac effects on humans.

Damiana (Turnera diffuse, syn. Turnera aphrodisiaca) is a shrub native to Central and South America. It belongs to the family Turneraceae.

Blooming with small yellow flowers, the shrub has an odor somewhat like chamomile, which is due to an oil present in the plant. The leaves have traditionally been made into a tea, which was used by native people of Central and South America for its reputed aphrodisiac effects.

Damiana is also a European name. In the country of Bulgaria it is simply a female version of Damian. In Greece the name Damiana refers to a person who is tame and subdued. Additionally, the name Damiana is somewhat common in Latino/Spanish locations.

In herbal medicine, Damiana is used to treat conditions ranging from coughs, to constipation, to depression. The herbal supplement is reputed to help with energy, emphysema, low estrogen, frigidity, hot flashes, impotency, infertility, menopause, Parkinson's disease, PMS, inflammation of prostate, Lou Gehrig's disease, and more dealing with reproductive organs in both males and females.

Damiana is obtained from the shrub Turnera diffusa, which is native to the U.S. Southwest and northern Mexico. The inhabitants of this region have used Damiana for many years as a remedy for nervous disorders, and as a tonic and aphrodisiac. Damiana seems to have a positive toning effect on both the nervous system and sexual organs.

Chocolate—The word “chocolate” comes from the Aztecs of Mexico, and is derived from the Nahuatl word xocolatl (http://www.yourdictionary.com/wotd/wotd), which is a combination of the words, xocolli, meaning “bitter”, and atl, which is “water”. The Aztecs associated chocolate with Xochiquetzal, the Goddess of Fertility. Chocolate is also associated with the Mayan god of fertility. Mexican philologist Ignacio Davila Garibi, proposed that “Spaniards had coined the word by taking the Mayan word chocol and then replacing the Mayan term for water, haa, with the Aztec one, atl.” (http://www.theclevermouse.com/belovedbeans_public/coffeehistory.html). However, it is more likely that the Aztecs themselves coined the term (http://www.sacofoods.com/foodofthegods.html), having long adopted into Nahuatl the Mayan word for the “cacao” bean; the Spanish had little contact with the Mayans before Cortés' early reports to the Spanish King of the beverage known as xocolatl (http://www.news.cornell.edu/releases/Oct98/chocolate.cacao.hrs.html). However, Micheal D. Coe, professor Emeritus of Anthropology and Curator Emeritus in the Peabody Museum of Natural History at Yale University, and coauthor of the book The True History of Chocolate, argues that the word xocolatl appears in “no truly early source in the Nahuatl language or in Aztec culture.”

Chocolate has been used solely as a drink for nearly all of its history. The earliest record of using chocolate pre-dates the Mayans. Chocolate residue has been found in pottery dating to 1100 BC from Honduras (http://www.nhm.ac.uk/research-curation/projects/sloane-herbarium/hanssloane.htm) and 600-400 BC from Belize. The chocolate residue found in an early classic ancient Mayan pot in Rio Azul, northern Guatemala, suggests that Mayans were drinking chocolate around 400 A.D. In the New World, chocolate was consumed in a bitter, spicy drink called xocoatl, and was often flavored with vanilla, chile pepper, and achiote, (which is known today as annatto). Xocoatl was believed to fight fatigue, a belief that is probably attributable to the theobromine content. Other chocolate drinks combined it with such edibles as maize starch paste (which acts as an emulsifier and thickener), various fruits, and honey. In 1689 noted physician and collector Hans Sloane, developed a milk chocolate drink in Jamaica which was initially used by apothecaries, but later sold by the Cadbury brothers (Athena Review Vol. 2, no. 2 A Brief History of Chocolate, Food of the Gods).

Chocolate was also an important luxury good throughout pre-Columbian Mesoamerica, and cacao beans were often used as currency (http://www.sustainabletimes.ca/articles/chocolate.htm). For example, the Aztecs used a system in which one turkey cost one hundred cacao beans and one avocado was worth three beans.

Romantic lore commonly identifies chocolate as an aphrodisiac. The reputed aphrodisiac qualities of chocolate are most often associated with the simple sensual pleasure of its consumption. More recently, suggestion has been made that serotonin and other chemicals found in chocolate, most notably phenethylamine, can act as mild sexual stimulants. While there is no firm proof that chocolate is indeed an aphrodisiac, giving a gift of chocolate to one's sweetheart is a familiar courtship ritual.

Tang-kuei (danggui) is a long root that is known as a blood-nourishing agent. It is highly used among women because tang-kuei will help regulate uterine blood flow and contraction, but when it is in complex formulas it can be used by both men and women to nourish the blood, moisten the intestines, improve the circulation, calm tension and relieve pain. Tang-kuei can be made as tea or cooked with chicken to make soup and the taste is quite strong”.

Of the above substances the ones that are purported or rumored to have mild aphrodisiac effects on males are Maca and Yohimbine. Saw Palmetto has been reported to have the side effect of causing erectile dysfunction.

Of the above substances the ones that are purported or rumored to have mild aphrodisiac effects on females are Damiana and Maca.

Numerous approaches have been taken in attempts to treat impotence. These approaches include the use of external or internally implanted penile prosthesis. (See, e.g., U.S. Pat. No. 5,065,744, to Zumanowsky). A variety of drugs and methods for administering drugs have also been used in attempts to treat impotence. For example, U.S. Pat. No. 3,943,246 to Sturmer addresses treatment of impotence in men by buccal and peroral administration of daily doses of 300-1500 international units (I.U.) of oxytocin or daily divided doses of 150-250 I.U. of desamino-oxytocin. The patent discloses the buccal administration of 100 I.U. three times a day for 14 days, resulting in improvement of impotentia erectionis in 12 of the 16 patients treated.

U.S. Pat. No. 4,530,920 to Nestor et al. discloses that the administration of nonapeptide and decapeptide analogs of luteinizing hormone releasing hormone agonists may be useful in the induction or enhancement of sexual behavior or therapy for impotence or frigidity. Nestor et al. suggest numerous routes of administration of the analogs including buccal, sublingual, oral, parenteral (including subcutaneous, intramuscular, and intravenous administration), rectal, vaginal, and others.

U.S. Pat. No. 4,139,617 to Grunwell et al. suggests buccal and other routes of administration of 19-oxygenated-androst-5-enes for the endocrine mediated enhancement of the libido in humans.

U.S. Pat. No. 4,863,911 to Anderson et al. discloses methods for treating sexual dysfunction in mammals using a biooxidizable, blood-brain barrier penetrating estrogen derivative. One of the purported objects of the Anderson et al. invention is the treatment of “psychological impotence” in males. Test results showed that the drugs used in the study stimulated mounting behavior, intromission, and mount latency in castrated rats.

A number of publications have proposed the use of various vasodilators for the treatment of impotence in males. Attempts to utilize vasodilators for the treatment of impotence were prompted by the fact that a significant percentage of cases of impotence were noted to be vasculogenic, i.e. resulting from vascular insufficiency.

Voss et al., U.S. Pat. No. 4,801,587, issued Jan. 31, 1989, discloses the use of an ointment containing a vasodilator and a carrier agent for topical application to the penis of impotent men. The Voss et al. patent also describes application of such an ointment into the urethra of the penis using a catheter as well as a multi-step regimen for applying a vasodilator to the skin of the penis. In addition, Voss et al. proposes the surgical removal of a portion of the fibrous sheath surrounding the corpora cavernosum, thereby facilitating the penetration of a vasodilator-containing ointment into the corpora cavernosum. Vasodilators disclosed for use by Voss et al. include papaverine, hydralazine, sodium nitroprusside, phenoxybenzamine, and phentolamine. The Voss et al. patent, however, provides no information regarding the actual efficacy of the treatments proposed or the nature of the response to such treatments.

U.S. Pat. No. 4,127,118 to Latorre describes treating male impotence by direct injection of the vasodilating drugs into the corpus cavernosum and the corpus spongiosum of the penis using a syringe and one or more hypodermic needles. More particularly, the Latorre patent proposes the intracavernosal and intraspongiosal injection of sympathomimetic amines such as nylidrin hydrochloride, adrenergic blocking agents such as tolazoline hydrochloride, and direct acting vasodilators such as isoxsuprine hydrochloride and nicotinyl alcohol.

Brindley, G. S. (Br. J. Pharmac. 87:495-500, 1986) disclosed that, when injected directly into the corpus cavernosum using a hypodermic needle, certain smooth muscle relaxing drugs including phenoxybenzamine, phentolamine, thymoxamine, imipramine, verapamil, papaverine, and naftidrofuryl caused erection. This study noted that injection of an “appropriate dose of phenoxybenzamine or papaverine is followed by an unrelenting erection lasting for hours.” Injection of the other drugs studied, induced erections lasting from about 11 minutes to about 6.5 hours.

Zorgniotti et al., J. Urol. 133:39-41 (1985) demonstrated that the intracavernosal injection of a combination of papaverine and phentolamine could result in an erection in otherwise impotent men. Similarly, Althof et al. (J. Sex Marital Ther. 17(2): 101-112 (1991) reported that intracavernosal injection of papaverine hydrochloride and phentolamine mesylate resulted in improved erectile ability in about 84% of patients injected. However, in that study the dropout rate was 57%, fibrotic nodules developed in 26% of the patients, 30% of the patients developed abnormal liver function values, and bruising occurred in 19% of the patients.

Other studies describing intracavernosal injection of drugs using hypodermic needles for the treatment of impotence include: Brindley, J. Physiol. 342:24 P (1983); Brindley, Br. J. Psychiatr. 143:312-337 (1983); Virag, Lancet ii:978 (1982); and Virag, et al., Angiology 35:79-87 (1984).

While intracavernosal injection may be useful for inducing erections in impotent men, the technique has numerous drawbacks. Obvious drawbacks include pain, risk of infection, inconvenience and interference with the spontaneity of the sex act. Priapism (prolonged and other painful erection) also appears to be a potential problem when using injection methods. See, e.g. Brindley, (1986). Another problem arising in some cases of intracavernosal injection involves the formation of fibrotic lesions in the penis. See, e.g., Corriere, et al., J. Urol. 140:615-617 (1988) and Larsen, et al., (J. Urol. 137:292-293 (1987).

Phentolamine, which has been shown to have the potential to induce erection when injected intracavernosally, has also been the subject of oral administration to test its effects in men having non-specific erectile insufficiency (Gwinup, Ann. Int. Med. Jul. 15, 1988, pp. 162-163.) In that study, 16 patients ingested either a placebo or a 50 mg orally administered dose of phentolamine. Eleven of the 16 patients (including three placebo-treated patients) became tumescent, became more responsive to sexual stimulation, and were able to achieve an erection sufficient for vaginal penetration after waiting 1.5 hours to attempt intercourse.

Sonda et al. (J. Sex & Marital Ther. 16(1): 15-21 (year), reported that Yohimbine ingestion resulted in subjective improvement in erectile ability in 38% of impotent men treated, but only 5% of the treated patients reported complete satisfaction.

Zorgniotti et al, PCT/US94/09048, describes the transmucosal administration of a variety of vasodilators including phentolamine mesylate for modulating the human sexual response.

U.S. Pat. No. 4,885,173, to Stanley et al., discloses methods of administering drugs having cardiovascular or renal vascular activity through use of a lollipop assertedly facilitating drug absorption through the mucosal tissues of the mouth, pharynx, and esophagus. The Stanley et al. patent discloses that a large number of lollipop-administered drugs may improve cardiovascular function including drugs exhibiting direct vasodilating effects, including calcium channel blockers, beta-adrenergic blocking agents, serotonin receptor blocking agents, angina blocking agents, other anti-hypertensive agents, cardiac stimulating agents, and agents, which improve renal vascular function.

U.S. Pat. No. 5,059,603 to Rubin describes the topical administration to the penis of isoxsuprine and caffeine, and nitroglycerine and caffeine along with suitable carrier compounds for the treatment of impotence.

U.S. Pat. No. 5,902,593 to Kent, et al., discloses a topically applied aphrodisiac dispersed in a manually applied vehicle, which substantially increases tissue sensation. The principal ingredient is benzalkonium chloride in a water-soluble gel, which includes sorbitol, glycerin, hydroxethylcellulose and propylene glycol.

U.S. Pat. No. 5,281,423 to Reilly discloses a method of heightening sexual desire of an adult human female comprising administering to an adult human female in need of said treatment, an effective amount of hydriodic acid syrup, comprising hydriodic acid, water and dextrose.

U.S. Pat. No. 3,943,246 to Sturmer discloses a method of treating impotency in a human male in need of said treatment, which comprises administering oxytocin at a dosage of 300 to 1500 I.U. daily.

The above issued patents comprise means, which are either considered the ingestion of a drug orally or by other means. The drugs are not a good solution since it is highly probable that prolonged use will result in side effects that are unwanted.

Natural remedies for which patents have been issued include:

U.S. Pat. No. 6,803,060 to Reyes discloses a composition for boosting libido of an individual, said composition consisting essentially of an effective amount 667 mg Tribulus, 427 mg Muria Puama, 352 mg, Catuba Bark, 127 mg L-Arginine, 60 mg Avena Sativa, and 37 IU Vitamin E.

Reyes further discloses a spray composition for boosting libido of an individual, said composition consisting essentially of 35 mg Tribulus terrestris, 30 mg Epimedium sagattatium, 10 mg Muria Puama, 10 mg Serenoa reopens, 10 mg Chrysin, and 4 mg 5-Androstenediol.

U.S. Pat. No. 6,093,421 to DeLuca et al. discloses a process of increasing testosterone levels in a man comprising oral administering to a man in need of such a treatment, an effective amount of a composition containing Maca and antler.

### Aphrodisiac Drugs

### Testosterone

Libido is clearly linked to levels of sex hormones, particularly testosterone. (R. Shabsigh (1997). “The effects of testosterone on the cavernous tissue and erectile function”. World J. Urol). When reduced sex drive occurs in individuals with relatively low levels of testosterone (e.g., post-menopausal women or men over age 60), testosterone supplements will often increase libido. Approaches using a number of precursors intended to raise testosterone levels have been effective in older males (Brown, G. A.; Vukovich M D, Martini E R, Kohut M L, Franke W D, Jackson D A, King D S. (2001). “Effects of androstenedione-herbal supplementation on serum sex hormone concentrations in 30- to 59-year-old men”. Int J Vitam Nutr Res.), but have not fared well when tested on other groups (Brown, G. A.; Vukovich M D, Reifenrath T A, Uhl N L, Parsons K A, Sharp R L, King D S. (2000). “Effects of anabolic precursors on serum testosterone concentrations and adaptations to resistance training in young men.”. Int J Sport Nutr Exerc Metab.).

### Yohimbine

Yohimbine is the main alkaloid of Yohimbe. As a weak MAO inhibitor and alpha-adrenergic antagonist, Yohimbine may increase genital blood-flow and sexual sensitivity for some people (Adeniyi, A. A.; Brindley G S, Pryor J P, Ralph D J. (2007). “Yohimbine in the treatment of orgasmic dysfunction”. Asian J Androl. and Kovalev, V. A.; Koroleva S V, Kamalov A A. (2000). “Pharmacotherapy of erectile dysfunction.”. Urologiia)

### Bremelanotide

Bremelanotide, formerly known as PT-141, is currently undergoing clinical trials for the treatment of sexual arousal disorder and erectile dysfunction. It is intended for both men and women. Preliminary results are encouraging (King, S. H.; Mayorov A V, Balse-Srinivasan P, Hruby V J, Vanderah T W, Wessells H. (2007). “Melanocortin receptors, melanotropic peptides and penile erection.”. Curr Top Med Chem.)

### PEA

There is some debate in lay circles as to whether a chemical called phenylethylamine present in chocolate is an aphrodisiac. This compound, however, is quickly degraded by the enzyme MAO such that significant concentrations do not reach the brain.

### Other Drugs

Stimulants affecting the dopamine system such as cocaine and amphetamines (e.g. Methamphetamine, aka Crystal meth) are frequently associated with hyperarousal and hypersexuality, though both may impair sexual functioning, particularly in the long term.

### Drugs Not Considered Aphrodisiacs

Psychoactive substances like alcohol, cannabis (Cannabis Puts Females in the Mood for Love. Mark Henderson, The Times (29 Jan. 2001)) and MDMA are not aphrodisiacs in the strict sense of the definition, but they can be used to increase sexual pleasure and to reduce sexual inhibition.

Anti-erectile dysfunction drugs, such as Viagra and Levitra, are not considered aphrodisiacs because they do not have any mood effect.

### Aphrodisiac Foods and Herbs

Some natural items purported to be aphrodisiacs when ingested (not at all exhaustive): Arugula (Rocket), Balut, Chocolate, Damiana, Eggs, Eurycoma longifolia (Ang, H. H.; M. K. Sim (1997). “Eurycoma longifolia Jack enhances libido in sexually experienced male rats”. Exp Anim and Ang, H. H.; Lee K L, Kiyoshi M. (2004). “Sexual arousal in sexually sluggish old male rats after oral administration of Eurycoma longifolia Jack.”. J Basic Clin Physiol Pharmacol.), Ginkgo biloba (McKay, D. (2004). “Nutrients and botanicals for erectile dysfunction: examining the evidence”. Altern Med Rev and Cohen, A. J.; Bartlik B. (1998). “Ginkgo biloba for antidepressant-induced sexual dysfunction.”. J Sex Marital Ther.), Ginseng (Sandroni, P. (October 2001). “Aphrodisiacs past and present: a historical review.”. Clin Auton Res. 11 (5): 303-7. and Murphy, L. L.; Lee T J. (2002). “Ginseng, sex behavior, and nitric oxide.”. Ann N Y Acad Sci.), Honey, Kelp, Maca (Gonzales, G. F.; Córdova A, Vega K, Chung A, Villena A, Góñez C. (2003). “Effect of Lepidium meyenii (Maca), a root with aphrodisiac and fertility-enhancing properties, on serum reproductive hormone levels in adult healthy men.”. J Endocrinol and Gonzales, G. F.; Córdova A, Vega K, Chung A, Villena A, Góñez C, Castillo S. (2002). “Effect of Lepidium meyenii (MACA) on sexual desire and its absent relationship with serum testosterone levels in adult healthy men.”. Andrologia), Oat, Oysters, Shilajit, Socratea exorrhiza, Spanish Fly (cantharidin) (Karras, D. J.; Farrell S E, Harrigan R A, Henretig F M, Gealt L. (1996). “Poisoning from “Spanish fly” (cantharidin).”. Am J Emerg Med), Tribulas terrestrias (Gauthaman, K.; A. P. Ganesan, R. N. Prasad. (2003). “Sexual effects of puncturevine (Tribulus terrestris) extract (protodioscin): an evaluation using a rat model.”. J Altern Complement Med and Gauthaman, K.; P. G. Adaikan, R. N. Prasad. (2002). “Aphrodisiac properties of Tribulus Terrestris extract (Protodioscin) in normal and castrated rats.”. Life Sci. and Neychev, V. K.; V. I. Mitev (2005). “The aphrodisiac herb Tribulus terrestris does not influence androgen production in young men.”. J Ethnopharmacol.), and Walnut Oil.

Some newly introduced exotic fruits or vegetables often acquire such a reputation, at least until they become more familiar; for example:

### Artichokes, Asparagus, Strawberries, Tomatoes, Truffles, Turtle Eggs, Mangoes, and Mamey Sapote.

The problem with the herbal treatments is that they simply are not as powerful as the drugs. This problem is addressed by the proposed invention.

### DETAILED DESCRIPTION OF THE INVENTION

The proposed invention is not a vasodilator. It is a combination of herbs with an additive catalyst substance, which promotes the herbs to stimulate the glands in the brain generating a psychotropic effect causing the body to generate its own unique chemistry specific to the augmentation and enhancement of sexual function. In the absence of the catalyst substance the herbs simply will not have the effects reported, thus the invention would be inoperative. The catalyst substance by itself has no aphrodisiac properties. It is therefore unobvious to combine it with said herbs. The unexpected result can be measured in the following way: If an herbal aphrodisiac is used by an individual the aphrodisiac effects, if measured on a scale of one to one hundred qualitatively by the user, are reported to have a score around thirteen to eighteen. The user will often make comments like, “I think it might be doing something”. If on the other hand the herbal aphrodisiac is mixed with an effective amount of the catalyst substance, the user reports the score of the effectiveness to be from eighty to one hundred on a scale of one to one hundred. The catalyst substance has to be intimately mixed with the herbs and is the critical key to the function of the invention. The catalyst substance is omega oil. In particular hemp seed oil. The hemp seed oil having all of the THC removed.

The herbs are combined with the omega oils in the form of extracts.

Examples of how the essence of herbs are extracted from the plant with hot water are organized in the following discussion. These are essentially herbal teas. Typically, one ounce by weight (about a cup by volume) of dried herb is placed in a quart jar, which is then filled to the top with boiling water, tightly lidded and allowed to steep for 4-10 hours. After straining, a cup or more is consumed, and the remainder chilled to slow spoilage. Drinking 2-4 cups a day is usual. Since the minerals and other phytochemicals in nourishing herbs are made more accessible by drying, dried herbs are considered best for infusions.

Herbal tinctures are concentrates made by dipping the plant into alcohols, the resulting fluid containing the desired ingredients of the herb. Steeping a medicinal plant in alcohol extracts the alcohol-soluble principles into a liquid form that can be stored for long periods. Different concentrations of alcohol are used to extract different constituents of the plants. For example; resins require high alcohol content and sugars usually require low alcohol content for optimal extraction.

There are many schools of thought about tincture making. In the traditional view an herb is either steeped once (single maceration) or more than once. In a double maceration the mark (or used plant material) is removed and replaced by a new batch (using the same alcohol) thus increasing the strength of the tincture. Sometimes the mark is then ashed (burnt until ash) and added back in which increase the amount of some minerals in the tincture.

In the scientific model tincture strengths are measured by a ratio of herb to alcohol (1:5 and 1:2 are the most common where the 1:2 is the stronger tincture). Many tinctures use a combination of vegetable glycerine and alcohol to extract, which changes the compounds that are extracted.

Fluid extracts are stronger than herbal tinctures, and can be preserved with alcohol or glycerin. They are just highly concentrated tinctures, made by distilling off some of the alcohol used in the tincture process. The final result is a liquid plant compound that can be 40 times more potent than a tincture.

Glycerates are herbal extracts that use glycerin as the sole extractant. They are very different and often have completely different medicinal properties than alcohol extracts. Tinctures or fluid extracts that are alcohol free should have the alcohol removed after the extraction process and replaced with glycerin which then acts just as the preservative.

In general, for the purpose of clarifying the optimum conditions of the proposed invention we will provide effective amounts of herbs by way of extracts. The extracts will be quantified by their volume, and the process by which the extract was created will be classified by the weight of herbal plant material to the volume of extractant used to capture the valuable essences from the plant. For example, a volume V of a 1 wght:2 vol process alcohol liquid extract of Maca would mean the following (numbers are used to add clarity); 1000 grams of Maca plant was placed in 2000 milliliters of alcohol and allowed to sit for a period of time, at least equal to the extract maturation time, during which time the alcohol extracts essence from the plant in the form of alkaloids. The maturation time of the steeping is the time wherein the maximum amount of alkaloids have been extracted from the plant material and dissolved into the fluid, at which point the fluid has reached saturation. Saturation meaning that the fluid cannot receive any more plant material alkaloids and if more alkaloids are extracted from the plant they cannot dissolve into the fluid, but rather they precipitate out as solids onto the floor of the container. The remaining plant material and any remaining solid precipitants are discarded and the resulting fluid is the liquid extract of value. This would constitute a 1 wght:2 vol process alcohol extract, with alcohol as a carrier, and a volume that will be slightly greater than the 1000 ml of pure alcohol that started the process. Other words that are equivalent to describe the extract would be, a volume V of mature, fully saturated herbal extract which originated from a 1 wght:2 vol alcohol extraction process wherein the alcohol remained as the carrier and the preservative.

An alcohol herbal extract can be made alcohol free by removing the alcohol and adding an effective amount of glycerin as a carrier and a preservative. We shall refer to such an extract as an alcohol herbal extract made alcohol free and further including glycerin as a carrier and preservative. More exact words might be, a volume V of mature, fully saturated herbal extract which originated from a 1 wght:2 vol alcohol extraction process wherein said alcohol was discarded and replaced with an effective amount of glycerin.

An extract that originated from a glycerin extraction process would be described as a mature, fully saturated herbal extract which originated from a 1 wght:2 vol glycerin extraction process wherein the glycerin remained as the carrier and the preservative. This is just a way of filling space.

In one embodiment of the proposed invention a liquid plant compound is combined with an effective amount of omega oils for the enhancement of libido. The plant compound is derived from herbs, which are known to have aphrodisiac effects on males.

In one embodiment of the proposed invention, a liquid plant compound is combined with an effective amount of omega oils for the enhancement of libido. The plant compound is derived from herbs, which are known to have aphrodisiac effects on males. Said herbs being selected from the group consisting of Maca, Saw Palmetto, and Yohimbine.

In one embodiment of the proposed invention, a liquid plant compound is combined with an effective amount of omega oils for the enhancement of libido. The plant compound is derived from herbs, some of which are known to have aphrodisiac effects on females.

In one embodiment of the proposed invention, a liquid plant compound is combined with an effective amount of omega oils for the enhancement of libido. The plant compound is derived from herbs, some of which are known to have aphrodisiac effects on females. Said herbs being selected from the group consisting of Maca, Damiana, and Tang Kuei.

In one embodiment of the proposed invention, a liquid plant compound is combined with an effective amount of omega oils for the enhancement of libido. The plant compound is derived from herbs, which are known to have aphrodisiac effects on males. Said herbs being selected from the group consisting of Maca and Yohimbine.

In one embodiment of the proposed invention, a liquid plant compound is combined with an effective amount of omega oils for the enhancement of libido. The plant compound is derived from herbs, which are known to have aphrodisiac effects on males. Said herbs being selected from the group consisting of Maca and Yohimbine. Yet, further included is an effective amount Saw Palmetto, the amount of Saw Palmetto, being at least as much as the greatest amount of either Yohimbine or Maca. This mixture is unusual in that the large amount of Saw Palmetto is in danger of causing erectile dysfunction in males. Yet, in the combination of omega oils in the proposed invention the Saw Palmetto in large amounts has the effect of greater enhancement of the aphrodisiac effects of the other herbs.

In one embodiment of the proposed invention, a liquid plant compound is combined with an effective amount of omega oils for the enhancement of libido. The plant compound is derived from herbs, which are known to have aphrodisiac effects on females. Said herbs being selected from the group consisting of Damiana, Maca, and Tang-Kuei.

In one embodiment of the proposed invention, a liquid plant compound is combined with an effective amount of omega oils for the enhancement of libido. The plant compound is derived from herbs, which are known to have aphrodisiac effects on males. Yet, further included is an effective amount of chocolate.

In one embodiment of the proposed invention, a liquid plant compound is combined with an effective amount of omega oils for the enhancement of libido. The plant compound is derived from herbs, some of which are known to have aphrodisiac effects on females. Yet, further included is an effective amount of chocolate.

In one embodiment of the proposed invention, a liquid plant compound is combined with an effective amount of omega oils for the enhancement of libido. The plant compound is derived from herbs, which are known to have aphrodisiac effects on males. Said herbs being selected from the group consisting of Maca and Yohimbine. Yet, further included is an effective amount of chocolate.

In one embodiment of the proposed invention, a liquid plant compound is combined with an effective amount of omega oils for the enhancement of libido. The plant compound is derived from herbs, which are known to have aphrodisiac effects on males. Said herbs being selected from the group consisting of Maca and Yohimbine. Yet, further included is an effective amount Saw Palmetto, the amount of Saw Palmetto being at least as much as the greatest amount of either Yohimbine or Maca. This mixture is unusual in that the large amount of Saw Palmetto is in danger of causing erectile dysfunction in males due to its reported side effects. Yet, in the combination of omega oils in the proposed invention the Saw Palmetto in large amounts has the effect of greater enhancement of the aphrodisiac effects of the other herbs. Yet, further included is an effective amount of chocolate.

In one embodiment of the proposed invention, a liquid plant compound is combined with an effective amount of omega oils for the enhancement of libido. The plant compound is derived from herbs, which are known to have aphrodisiac effects on females. Said herbs being selected from the group consisting of Damiana and Tang-Kuei, yet further included is an effective amount of Maca and chocolate.

In the above embodiments the effectual amounts of ingredients are present in a fluid, the total volume of the fluid aphrodisiac being Vₐ, the total volume of the herbal extract being Vₕ, and the volume of omega oils being Vₒ, such that Vₐ=Vₕ+Vₒ.

In the embodiments wherein the liquid herbal extract is composed of Maca and Yohimbine the total volume of the fluid aphrodisiac is Vₐ, the total volume of the herbal extract is Vₕ, and the volume of omega oils is Vₒ, such that Vₐ=Vₕ+Vₒ. The volume of the Maca extract is between 60% and 75% of Vₕ and the volume of the Yohimbine extract is between 25% and 40% of Vₕ.

In the embodiments wherein the liquid herbal extract is composed of Maca, Yohimbine, and Saw Palmetto the total volume of the fluid aphrodisiac is Vₐ, the total volume of the herbal extract is Vₕ and the volume of omega oils is Vₒ, such that Vₐ=Vₕ+Vₒ. The volume of the Maca extract is between 35% and 50% of Vₕ. The volume of the Yohimbine extract is between 25% and 30% of Vₕ. The volume of the Saw Palmetto extract is between 25% and 35% of Vₕ.

In the embodiments wherein the liquid herbal extract is composed of Maca, Damiana, and Tang-Kuei, the total volume of the fluid aphrodisiac is Vₐ, the total volume of the herbal extract is Vₕ, and the volume of omega oils is Vₒ, such that Vₐ=Vₕ+Vₒ. The volume of the Maca extract is between 35% and 50% of Vₕ. The volume of the Damiana extract is between 25% and 30% of Vₕ. The volume of the Tang-Kuei extract is between 25% and 35% of Vₕ.

In the above embodiments the liquid extracts are combined with effective amounts of omega oils. Again the total volume of the fluid aphrodisiac is Vₐ, the total volume of the herbal extract is Vₕ, and the volume of omega oils is Vₒ, such that Vₐ=Vₕ+Vₒ. The mixture is such that Vₕ is between 75% and 85% of Vₐ, and Vₒ is between 15% and 25% of Vₐ.

In the above embodiments wherein the liquid plant extract is combined with omega oils and chocolate, the chocolate can be a syrup. The volume of the chocolate syrup aphrodisiac mix is Vs. The volume of the fluid aphrodisiac is Vₐ. The volume of the chocolate syrup is V_{c}. The total volume of the chocolate syrup aphrodisiac mix is Vₛ, such that Vₛ=Vₐ+V_{c}. The mixture is such that Vₐ is between 20% and 25% of Vₛ and V_{c} is between 75% and 80% of Vₛ.

Thus the reader can see that there are many embodiments of the proposed invention. While the above description contains many specificities, these should not be construed as limitations on the scope of the invention, but rather as an exemplification of several preferred embodiments thereof.

One can see that the effectiveness of herbal aphrodisiacs is unexpectedly greatly enhanced by the combining of such herbs with effective amounts of omega oils, thus creating a new combination of substances, which can outperform the libido enhancing drugs that have been developed.

Accordingly the scope of the invention should be determined not by the embodiments illustrated, but by the appended claims and their legal equivalents.

What we claim is:

1. A method of enhancing the libido in an adult male comprising the administering of a composition, comprising an effective amount of an herbal aphrodisiac selected from the group consisting of those herbs, which are purported to enhance the libido in males, and an effective amount of omega oils.

2. The method of claim one wherein said herbal aphrodisiac is a mixture of Yohimbine and Maca.

3. The method of claim one wherein said herbal aphrodisiac is a mixture of Yohimbine and Maca, and further including an effective amount of Saw Palmetto.

4. The method of claim one wherein said herbal aphrodisiac is a mixture of Yohimbine and Maca, and further including an effective amount of Saw Palmetto, and the mixture of said herbs is within the amounts comprising 2 parts Maca, 0.5 to 1.5 parts Yohimbine, and 1 to 3 parts Saw Palmetto.

5. The method of claim one wherein said omega oil is in the form of hemp seed oil.

6. The method of claim one wherein said herbs are in the form of liquid herbal extracts.

7. The method of claim one wherein said herbs are in the form of liquid herbal extracts being mature, fully saturated extract which originated from a 1 wght:2 vol alcohol extraction process, wherein the alcohol remained as the carrier and the preservative.

8. The method of claim one wherein said herbs are in the form of liquid herbal extracts being mature, fully saturated extract which originated from a 1 wght:2 vol alcohol extraction process, wherein said alcohol was discarded and replaced with an effective amount of glycerin.

9. The method of claim one wherein said herbs are in the form of liquid herbal extracts being mature, fully saturated extract which originated from a 1 wght:2 vol glycerin extraction process, wherein the glycerin remained as the carrier and the preservative.

10. The method of claim one wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, the mixture being such that Vₕ is between 75% and 85% of Vₐ, and Vₒ is between 15% and 25% of Vₐ.

11. The method of claim one wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, the mixture being such that Vₕ is between 75% and 85% of Vₐ, and Vₒ is between 15% and 25% of Vₐ, and said liquid herbal extract being a fully saturated herbal extract which originated from a 1 wght:2 vol alcohol extraction process, wherein the alcohol remained as the carrier and the preservative.

12. The method of claim one wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, the mixture being such that Vₕ is between 75% and 85% of Vₐ, and Vₒ is between 15% and 25% of Vₐ, and said liquid herbal extract being a mature, fully saturated herbal extract which originated from a 1 wght:2 vol alcohol extraction process, wherein said alcohol was discarded and replaced with an effective amount of glycerin.

13. The method of claim one wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, the mixture being such that Vₕ is between 75% and 85% of Vₐ, and Vₒ is between 15% and 25% of Vₐ, and said liquid herbal extract being a mature, fully saturated herbal extract which originated from a 1 wght:2 vol glycerin extraction process wherein the glycerin remained as the carrier and the preservative.

14. The method of claim one wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, and said herbal extract being composed of Maca and Yohimbine, the volume of the Maca extract being between 60% and 75% of Vₕ and the volume of the Yohimbine extract being between 25% and 40% of Vₕ.

15. The method of claim one wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, and said herbal extract being composed of Maca and Yohimbine, the volume of the Maca extract being between 35% and 50% of Vₕ and the volume of the Yohimbine extract being between 25% and 35% of Vₕ, and the total fluid aphrodisiac mixture being such that Vₕ is between 75% and 85% of Vₐ, and Vₒ is between 15% and 25% of Vₐ.

16. The method of claim one wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, and said herbal extract being composed of Maca, Yohimbine, and Saw Palmetto, the volume of the Maca extract being between 35% and 50% of Vₕ and the volume of the Yohimbine extract being between 25% and 30% of Vₕ, and the volume of the Saw Palmetto extract being between 25% and 35% of Vₕ.

17. The method of claim one wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ and the volume of omega oils being Vₒ, such that Vₐ=Vₕ+Vₒ, and said herbal extract being composed of Maca, Yohimbine, and Saw Palmetto, the volume of the Maca extract being between 30% and 35% of Vₕ and the volume of the Yohimbine extract being between 20% and 25% of Vₕ, and the volume of the Saw Palmetto extract being between 20% and 30% of Vₕ, and the total fluid aphrodisiac mixture being such that Vₕ is between 75% and 85% of Vₐ, and Vₒ is between 15% and 25% of Vₐ.

18. The method of claim one wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, yet further included to said fluid aphrodisiac is an effective amount of liquid chocolate syrup wherein the volume of the chocolate syrup aphrodisiac mix is Vs, the volume of the fluid aphrodisiac being Vₐ, the volume of the chocolate syrup being V_{c}, the total volume of the chocolate syrup aphrodisiac mix being Vₛ, such that Vₛ=Vₐ+V_{c}, the mixture being such that Vₐ is between 20% and 25% of Vₛ and Vₐ is between 75% and 80% of Vₛ.

19. A method of enhancing the libido in an adult female comprising the administering of a composition, comprising an effective amount of an herbal aphrodisiac selected from the group consisting of those herbs, which are purported to enhance the libido in females, and an effective amount of omega oils.

20. The method of claim nineteen wherein said herbal aphrodisiac is a mixture of Maca and Damiana.

21. The method of claim nineteen wherein said herbal aphrodisiac is a mixture of Maca, Damiana, and Tang-Kuei.

22. The method of claim nineteen wherein said herbal aphrodisiac is a mixture of Maca, Damiana, and Tang-Kuei, and the mixture of said herbs is within the amounts comprising 2 parts Maca, 0.5 to 1.5 parts Damiana, and 1 to 3 parts Tang-Kuei.

23. The method of claim nineteen wherein said omega oil is in the form of hemp seed oil.

24. The method of claim nineteen wherein said herbs are in the form of liquid herbal extracts.

25. The method of claim nineteen wherein said herbs are in the form of liquid herbal extracts being mature fully saturated extract which originated from a 1 wght:2 vol alcohol extraction process, wherein the alcohol remained as the carrier and the preservative.

26. The method of claim nineteen wherein said herbs are in the form of liquid herbal extracts being mature fully saturated extract which originated from a 1 wght:2 vol alcohol extraction process, wherein said alcohol was discarded and replaced with an effective amount of glycerin.

27. The method of claim nineteen wherein said herbs are in the form of liquid herbal extracts being mature fully saturated extract which originated from a 1 wght:2 vol glycerin extraction process wherein, the glycerin remained as the carrier and the preservative.

28. The method of claim nineteen wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, the mixture being such that Vₕ is between 75% and 85% of Vₐ, and Vₒ is between 15% and 25% of Vₐ.

29. The method of claim nineteen wherein the effectual amounts of ingredients are present in the form of a fluid, the omega oil being a fluid, the herbs being present in the form of a liquid herbal extract the total volume of the fluid aphrodisiac being Vₐ, the volume of the liquid herbal extract being Vₕ and the volume of omega oils being Vₒ, such that Vₐ=Vₕ+Vₒ, and said herbal extract being composed of Maca and Damiana, the volume of the Maca extract being between 60% and 75% of Vₕ, and the volume of the Damiana extract being between 25% and 40% of Vₕ.

30. The method of claim nineteen wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, and said herbal extract being composed of Maca and Damiana, the volume of the Maca extract being between 35% and 50% of Vₕ, and the volume of the Damiana extract being between 25% and 35% of Vₕ, and the total fluid aphrodisiac mixture being such that Vₕ is between 75% and 85% of Vₐ, and Vₒ is between 15% and 25% of Vₐ.

31. The method of claim nineteen wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ and the volume of omega oils being Vₒ, such that Vₐ=Vₕ+Vₒ, and said herbal extract being composed of Maca, Damiana, and Tang-Kuei, the volume of the Maca extract being between 35% and 50% of Vₕ, and the volume of the Damiana extract being between 25% and 30% of Vₕ, and the volume of the Tang-Kuei extract being between 25% and 35% of Vₕ.

32. The method of claim nineteen wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, and said herbal extract being composed of Maca, Damiana, and Tang-Kuei, the volume of the Maca extract being between 30% and 35% of Vₕ, and the volume of the Damiana extract being between 20% and 30% of Vₕ, and the volume of the Tang-Kuei extract being between 20% and 25% of Vₕ, and the total fluid aphrodisiac mixture being such that Vₕ is between 75% and 85% of Vₐ, and Vₒ is between 15% and 25.0% of Vₐ.

33. The method of claim nineteen wherein the effectual amounts of ingredients are present in the form of a fluid, the omega oil being a fluid, the herbs being present in the form of a liquid herbal extract, the total volume of the fluid aphrodisiac being Vₐ, the volume of the liquid herbal extract being Vₕ, and the volume of omega oils being Vₒ, such that Vₐ=Vₕ+Vₒ, yet further included to said fluid aphrodisiac is an effective amount of liquid chocolate syrup wherein the volume of the chocolate syrup aphrodisiac mix is Vₛ, the volume of the fluid aphrodisiac being Vₐ, the volume of the chocolate syrup being V_{c}, the total volume of the chocolate syrup aphrodisiac mix being Vₛ, such that Vₛ=Vₐ+V_{c}, the mixture being such that Vₐ is between 20% and 25% of Vₛ and V_{c} is between 75% and 80% of Vₛ.

34. The method of claim one wherein said herbal aphrodisiac is a mixture of Maca and Tribulus Terrestrias.

35. The method of claim one wherein said herbal aphrodisiac is a mixture of Maca, Tribulus Terrestrias, and Saw Palmetto.

36. The method of claim one wherein said herbal aphrodisiac is a mixture of Maca and Epimidium.

37. The method of claim one wherein said herbal aphrodisiac is a mixture of Maca, Epimidium, and Saw Palmetto.
